(19) United States
(12) Patent Application Publication
(10) Pub. No.: US 2018/0296441 A1
(43) Pub. Date: Oct. 18, 2018

(54) Smart Medication Bottle with Pill Dispense Detection and Pill Counting

(71) Applicant: WiMedics Incorporated, San Diego, CA (US)

(72) Inventors: Masoud Roham, San Diego, CA (US); Mohammad Meysam Zargham, San Diego, CA (US)

(21) Appl. No.: 15/953,877

(22) Filed: Apr. 16, 2018

Related U.S. Application Data
(60) Provisional application No. 62/485,988, filed on Apr. 16, 2017.

(51) Int. Cl. A61J 7/04; A61J 1/03; A61J 7/02; G01N 29/12; G01N 29/46

(52) U.S. Cl. CPC A61J 7/0436; A61J 1/03; A61J 7/02; G16H 20/13; G01N 29/46; A61J 2200/30; A61J 2200/72; G01N 29/12

(57) ABSTRACT

A bottle system is presented including a bottle portion having electronics including a display, an accelerometer, an acoustic sensor, and a processor, the bottle portion configured to hold a quantity of pills, and a cap portion joinable to the bottle portion. The accelerometer and processor are configured to determine an acceleration of shaking of the bottle portion and cap portion with the quantity of pills contained therein, and further wherein the acoustic sensor, accelerometer, and processor are configured to determine a frequency spectrum of an acoustic signal obtained when the bottle portion and cap portion are shaken and determine an estimated quantity of pills based on the frequency spectrum.

This application claims the benefit of the earlier filed co-pending Provisional Patent Application Ser. No. 62/485,988, filed Apr. 16, 2017, the contents of which are incorporated herein by reference in the entirety.

### BACKGROUND

### I. Field

The present invention relates generally to the field of healthcare, and more specifically to devices that contain and/or dispense pills.

### II. Background

Lack of medication compliance is a problem costing in excess of $200 billion in the United States alone. Many patients with chronic conditions have difficulty adhering to prescribed therapies. In general, the more medications taken and the more times each day that patients must use various therapies, the more likely there will be a medication error. Medication errors can include failure to take a medication at an opportune time and failure to take a medication in its entirety.

Many studies that show that management of chronic diseases is less than ideal in spite of the great advances in medicine. Factors that have been implicated in disease management include poor compliance with medicine regimen because patients forget to take their medicines, frequent need to go the pharmacist for refills and education, need for frequent visit to the health care professional's office to monitor the treatment response and to make any required changes in medicine regimen, lack of adequate health education and inadequate reinforcement thereof, under or over dosing of medicine, altered dosing regimen, and incorrect administration of medicine (Kane, S. et al., Advanced Therapy for Inflammatory Bowel Disease; 2002:9-11). Even more worrisome is that patients frequently do not inform physicians of personal noncompliance with a medicine regimen. Physicians in such cases often conclude that patient's condition is not responding to the current medicine regimen and make changes in medicine dose, add or substitute another medicine. This results in unnecessary changes in patient's medicine regimen, including an increase in dosage not in accordance with the actual needs of the patient, but which in any set of circumstances can be detrimental to the health of the patient or even fatal. This practice also increases health care costs.

People take medicine for many different reasons. Sometimes the medication is taken for a short period of time. For example, a person with an ear infection may receive a prescription for a certain medicine that he is to take for a week or so. Other times the medication is taken for a long period of time, possibly for the foreseeable life of the patient. For example, a person with high blood pressure may take a certain medicine daily or even multiple times daily. People taking medicine for a long period of time may receive a prescription that enables them to receive a certain quantity of the medicine at a time. They then have the prescription refilled, up to a certain number of times. After, the prescription expires (all refills are used) the person, or someone caring for them (hereinafter referred to collectively and/or individually as user), may need to call the doctor or see the doctor to receive a refill of the prescription. Prescriptions in many cases involve pills distributed in bottles, typically plastic containers. A “pill” as used herein may include a prescription drug, an over-the-counter medication, a vitamin or nutritional supplement, caplet, capsule, or any other tablet like object which is designed to be ingested by the user.

The status of the prescription needs to be tracked to ensure that the prescription does not expire. A doctor's appointment may be necessary to renew the prescription or possibly modify the prescription. Depending on the type of insurance, the medication, and/or the pharmacy (e.g., brick-and-mortar, mail order) the prescription may need to be filled in advance of the time the medication will be needed. Accordingly, the timeline associated with the necessary steps required to get a prescription and receive the medication needs to be established in advance.

Several solutions to the problems associated with prescription drugs have been proposed. Prescription “vending machines” have been proposed. These devices contain a plurality of medications and dispense them at an appropriate time. Few of these devices have been commercialized since they are relatively expensive to manufacture and have limited capacity for various medications and due to the expense tend not to be feasible for home or even in-hospital use. The reliability of these devices in a remote setting is also questionable. The e-pill MD.2 Monitored Automatic Medication Dispenser (www.epill.com) is an example of such a device, although it only dispenses a single medication container.

Another category of medication management devices is an organizer/reminder device. Typically, these devices use small trays or compartments and are self-programmed by users to remind them to take medications at a specific time. Typically, the user fills the device as needed. Errors can result when users either self-program or self-fill such devices. These errors become more common as the complexity of the medication regimen increases. Conventional organizer/reminder devices do not prevent these kinds of errors, and an individual taking multiple prescriptions at different intervals can have difficulty when using such devices. Since these devices do not record medication usage, and are not connected to a support service, they can have a limited beneficial effect on the ability for a patient to adhere to her regimen.

A third category of medication management consists of using radio frequency (RF) tags incorporated into pill containers that employ sensing of prescription bottles, each of which has an RF tag associated therewith. These devices may result in a large number of pill containers being positioned on the sensing device. This results in difficulty for users of the devices since they have to place a large number of containers randomly on the device. Containers can become lost or RF tags can become dislodged from containers. An additional limitation of this approach is the need to fill a large number of medication containers with a number of different medications all taken at a specific time by the user. These containers must be filled with a high degree of accuracy and precision. Labeling of containers containing many medications may be difficult since the containers may not be large enough to hold a legible label listing required information for each medication in the pill container.

More recent innovation in the area of medication compliance includes incorporating sensory technology onto pill containers or into the caps of those containers. An example of this are devices that monitor when the cap of a prescription pill bottle has been removed. This information is stored electronically and may be uploaded to a data network using a remote docking station. This method is difficult with many medications, individual devices do not have a counting ability to determine the number of pills or doses remaining in the bottle. Such a lack of counting ability can result in false dispensing events and ultimate a lack of adherence to a medication schedule.

Another example of such a device uses a smart collar as a pill container cap. Such technological applications allow for the standard counting of pills remaining in the container based on the number of times that the cap is removed and replaced. However, such a device has no mechanism for counting the actual contents of the bottle, and thus the number of medications or pills left can he unknown.

Prior designs have attempted to use the closure of a medicine container as a surrogate marker for compliance. There are major disadvantages to designs that rely on medicine container cap removal as a measure of compliance. Medicine containers with caps allow access to the entire medicine supply during each dispensing event. Once the device recognizes the removal of the cap, any number of doses may be removed from the bottle without proper recognition, thus seriously compromising the device's ability to properly record compliance. Even more troublesome is the possibility that the cap device might not be reinstalled on the bottle. If not, the subsequent removal of pills from the bottle are unmonitored. No prior design provides the necessary reliability and inexpensive implementation as a viable alternative to today's plastic disposable containers that provides an enhanced level of safety for consumers. Such prior designs are inadequate in the ways described.

Therefore, there is a need for a pill dispensing design that addresses issues with the previous designs.

### SUMMARY

The following presents a simplified summary in order to provide a basic understanding of some aspects of the claimed subject matter. This summary is not an extensive overview and is not intended to identify key/critical elements or to delineate the scope of the claimed subject matter. Its sole purpose is to present some concepts in a simplified form as a prelude to the more detailed description that is presented later.

The systems, methods, and devices of the design each have several aspects, no single one of which is solely responsible for its desirable attribute. Aspects of the present design provide an apparatus for monitoring patient adherence to a medication regime. The apparatus generally includes a container for holding objects, a housing mountable at an opening of the container, and at least one detector.

According to the present design, there is provided an apparatus comprising a bottle portion comprising electronics including an accelerometer, an acoustic sensor, and a processor, the bottle portion configured to hold a quantity of pills, and a cap portion joinable to the bottle portion. The accelerometer and processor are configured to determine at least one of an acceleration of shaking of the bottle portion and cap portion, and a tilting of the bottle portion to a predetermined orientation evidencing removal of at least one pill from the bottle portion. The acoustic sensor, accelerometer, and processor are configured to determine a frequency spectrum of an acoustic signal obtained when the bottle portion and cap portion are shaken and determine an estimated quantity of pills based on the frequency spectrum.

According to another embodiment of the present design, there is provided a medication container bottle system comprising a bottle portion comprising a display and a processor and configured to receive and maintain a quantity of pills, and a cap portion joinable to the bottle portion, wherein the cap portion and bottle portion comprise at least two of (a) an accelerometer provided with the bottle portion, (b) an acoustic sensor provided with the bottle portion, (c) a magnet provided with the cap portion and a corresponding magnetic sensor provided with the bottle portion, (d) an optical transmitter/receiver and a corresponding optical reflector provided with the bottle portion, and (e) at least one capacitive sensing electrode provided proximate an opening on the bottle portion.

According to a further embodiment, there is provided a bottle system, comprising a bottle portion comprising electronics including a display, an accelerometer, an acoustic sensor, and a processor, the bottle portion configured to hold a quantity of pills, and a cap portion joinable to the bottle portion. The accelerometer and processor are configured to determine an acceleration of shaking of the bottle portion and cap portion with the quantity of pills contained therein, and further wherein the acoustic sensor, accelerometer, and processor are configured to determine a frequency spectrum of an acoustic signal obtained when the bottle portion and cap portion are shaken and determine an estimated quantity of pills based on the frequency spectrum.

To the accomplishment of the foregoing and related ends, certain illustrative aspects are described herein in connection with the following description and the annexed drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

FIG. 1A is a general depiction of a side view of a first embodiment of the present design showing a user interface display;

FIG. 1B is a general depiction of a side view opposite the side having the user interface display;

FIG. 2 is a general representation illustrating communication in one embodiment of the present design;

FIG. 3 depicts shaking of the bottle of the present design and information derived from such shaking;

FIG. 4 is a flow chart illustrating the combined signal detection paths of the accelerometer signal and acoustic signal resulting in an accurate detection of the number of remaining pills in the pill bottle;

FIG. 5A shows removal of the cap portion from the bottle portion of the bottle system

FIG. 5B illustrates operation upon removal of the magnetic cap in combination with accelerometer signal production to indicate a pill or medical item dispensing event;

FIG. 6 is a flow chart illustrating the use of acoustic excitation for detection of the number of pills remaining without the application of shaking;

FIG. 7 is a flow chart illustrating the application of the communication block to display the medication label on the user interface display by way of cellphone camera use;

FIG. 8 is a general depiction of the capacitive sensor detecting a user finger or other similar object to create a dispensing event without bottle tilt; and

FIG. 9 illustrates an alternate two cap version of the present design.

### DETAILED DESCRIPTION

In this document, the words “embodiment,” “variant,” and similar expressions are used to refer to particular apparatus, process, or article of manufacture, and not necessarily to the same apparatus, process, or article of manufacture. Thus, “one embodiment” (or a similar expression) used in one place or context can refer to a particular apparatus, process, or article of manufacture; the same or a similar expression in a different place can refer to a different apparatus, process, or article of manufacture. The expression “alternative embodiment” and similar phrases are used to indicate one of a number of different possible embodiments. The number of possible embodiments is not necessarily limited to two or any other quantity.

The word “exemplary” is used herein to mean “serving as an example, instance, or illustration.” Any embodiment or variant described herein as “exemplary” is not necessarily to be construed as preferred or advantageous over other embodiments or variants. All of the embodiments and variants described in this description are exemplary embodiments and variants provided to enable persons skilled in the art to make or use the invention, and not to limit the scope of legal protection afforded the invention, which is defined by the claims and their equivalents.

The word “pill” is used herein to mean “a capsule, pill, gel filled medicine, powered and compressed ingestible material, vitamins or any such similar object.” Any embodiment or variant described herein as “pill” is not necessarily to be construed as only pressed powder medicine.

When viewed in totality, the present design presents an overall subset of sensors on the system used to detect a quantity of objects within the bottle thus further enabling medication adherence. This subsystem can include a communication block or cloud-based platform to maintain connected communications to the medication bottle, a display for relaying medical/bottle content information to the user incorporated onto one side of the bottle, a cap for securing the contents of the bottle, a sensor or mechanical device incorporated into the cap, object removal detection sensors and mechanisms, and object counting sensors and mechanisms.

One aspect of the present design is a computer communication component offering connection to a network using a cellular phone. This component facilitates connection to a communication hub, such as a WiFi router, and a cellular tower. Such a connection permits cloud-based communications and rapid medication information updates, as well as easy user reminders for medication times.

Another aspect of the present design is detecting whether the pill bottle cap has been removed by use of a Hall magnetic sensor, or alternately a HAL sensor available from Micronas that employs Hall sensors. Another mechanism of detection of the pill cap removal uses a reflective optical sensor placed on the bottle. A third uses a mechanical switch.

Another aspect of the present design includes detecting when a pill has been dispensed from the bottle using capacitive sensing of a patient's finger or other such object which may enter the bottle to remove an object within. Such a sensor is designed to sense when a user is attempting to remove a pill, or a small number of pills, from the bottle with a finger without tipping the bottle to one side.

A further aspect of the present design includes detecting when a pill has been dispensed from the bottle using of a low power accelerometer. Such a mechanism detects when the pill has been tipped beyond a set angle thereby registering a dispensing event. As an additive application of the accelerometer, a system wide wake-up can be employed by detecting any movement of the pill bottle from a static position.

Another aspect of the present design includes detecting the quantity of objects remaining or contained in the pill bottle using acoustic signal detection. Such detection can be done by acoustic excitation such as a buzzer, or by shaking of the pill bottle and detecting the frequency spectrum and amplitude of the acoustic signal. Such a signal can then be used to determine the quantity of pills in the pill bottle.

Sensors used in or with the device are of particular note. Such a device can provide precise medication adherence as well as real time positive feedback information to the user and any medical providers working with the user. One feature described herein distinguishes between events of the bottle cap being opened and closed without removing a pill out versus one or more pills being removed from the bottle. A user may open a pill bottle and close the bottle to check on and identify the pills without physically removing any pills. The present design described here identifies this scenario and prevents false pill dispensing detection.

A further feature is the ability of the bottle to distinguish between how pills are taken from the bottle, pills taken by reaching into the bottle versus pills taken through tilting. This provides a broader range of possible detection methods contrasted against a broader number of potential human interactions with the bottle.

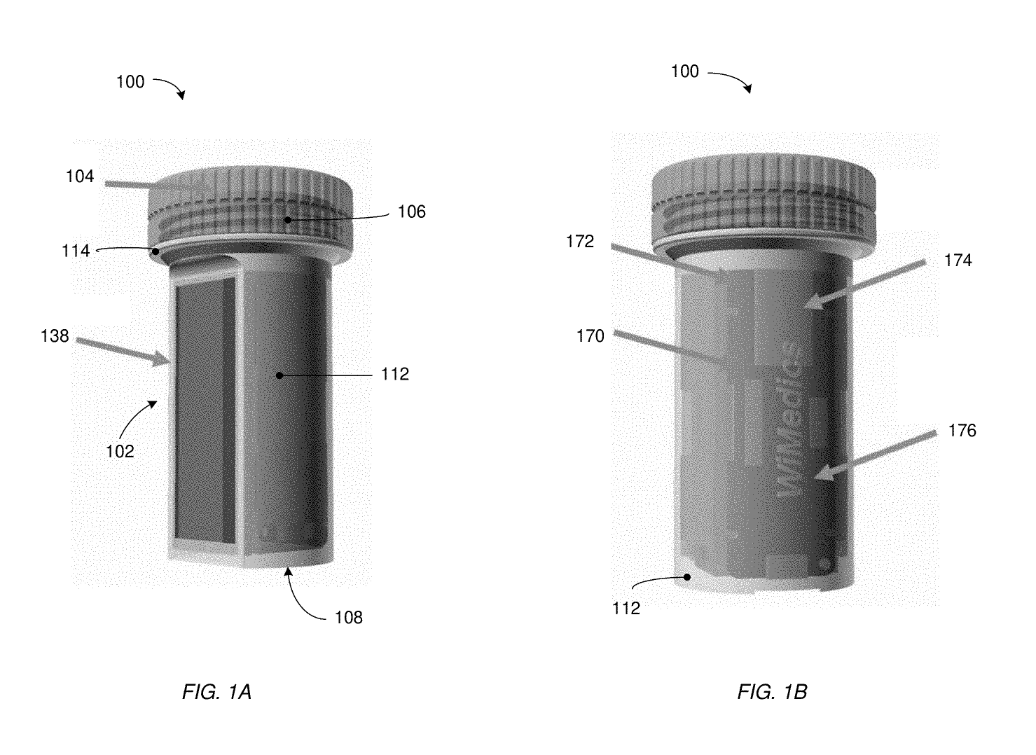

FIG. 1A shows one basic embodiment of the bottle system. The bottle system 100 externally consists of the bottle cap 104, the bottle base 108, the bottle wall exterior 112, the bottle collar 114, and the threaded end 106 of the bottle 102, where the threaded end 106 is partially obscured by the bottle cap in FIG. 1. The bottle cap 104 mounts to the top of the threaded end 106 of the bottle. A magnet (not shown) may be provided with or installed on the cap.

As shown in FIG. 1A, the bottle wall exterior 112 is mostly circumferential with one flat area on the circumference. However, this is one embodiment. The bottle wall exterior 112 does not need to be circumferential, alternate embodiments can be cuboid, rhomboid, or any other three-dimensional shape capable of containing pills. While the bottle wall exterior 112 can be of a number of geometric configurations, a requirement of the bottle 102 design is that a bottle aperture 110 or opening (not shown in this view) is created and located opposite the bottle base 108.

The flat area of FIG. 1A corresponds to the user interface display 138. The user interface display 138 does not need to be flat and may take a curved shape or other shape consistent with the bottle geometry selected by the end manufacturer. The user interface display partially extends the length of the bottle cap 104 of the bottle to the bottle base 108. The user interface display 138 allows for display of bottle content, medication times, medication strength, refills remaining, additional medication instructions such as taking medication with food, and any potential contraindications of the medication.

Shown in FIG. 1B is a rear view of the bottle wail exterior 112 showing the internal electronics of the bottle system 100. The internal electronics of the bottle system 100 are contained between the bottle wall interior 118 (not specifically identified in this view) and the bottle wail exterior 112. Thus, a double wall cavity 140 (not shown) is created to house the internal electronics, which in one embodiment may include electronics 170, magnetic sensor or Hall detector 172, communication module 174, and a microphone 176 couple to the interior of double wall cavity 140. This allows for sanitary conditions to be maintained with respect to the medication and a dust free environment maintained with respect to the electronics.

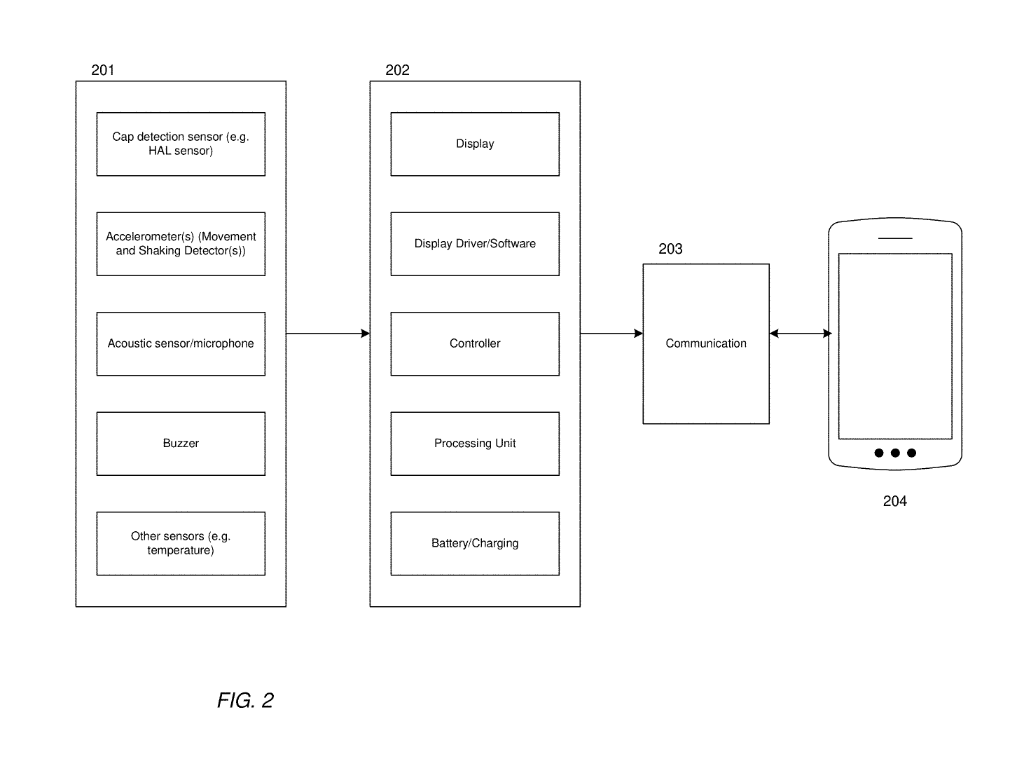

FIG. 2 is a broad, general flowchart representation of communication flow from the various sensors to physical devices to a communication block or network and to a user device for purposes of monitoring or otherwise interacting with the information received. FIG. 2 calls out sensors 201 which may include, for example, Hall detector 172, accelerometers 126, acoustic sensor 124, which may he a surface acoustic sensor, temperature sensor 144, and acoustic generator 146 (none shown in this view) that operate alone or in tandem to produce real time data for bottle system 100. Such real time data can then be displayed on the user interface display 138. The user interface display 138 includes hardware for displaying relevant information and the bottle system 100 includes supporting software and at least one processor typically used in informational displays. As shown in FIG. 2, bottle electronics 202 may include a display, display driver, controller, processing unit, and power, such as in the form of a battery or charging capability. The sensing data or sensed data can subsequently be sent through a communication block 203, which may comprise means of communication feasible under the circumstances, including but not limited to Bluetooth, Wifi, cellular, data network, Internet, or other electronic communication means, including combined communication means. The user or some authorized and authenticated individual may receive information on his or her personal device, such as a smartphone, shown as device 204. Information may also be provided from the user device via communication block 203 to the bottle electronics 202 of bottle system 100.

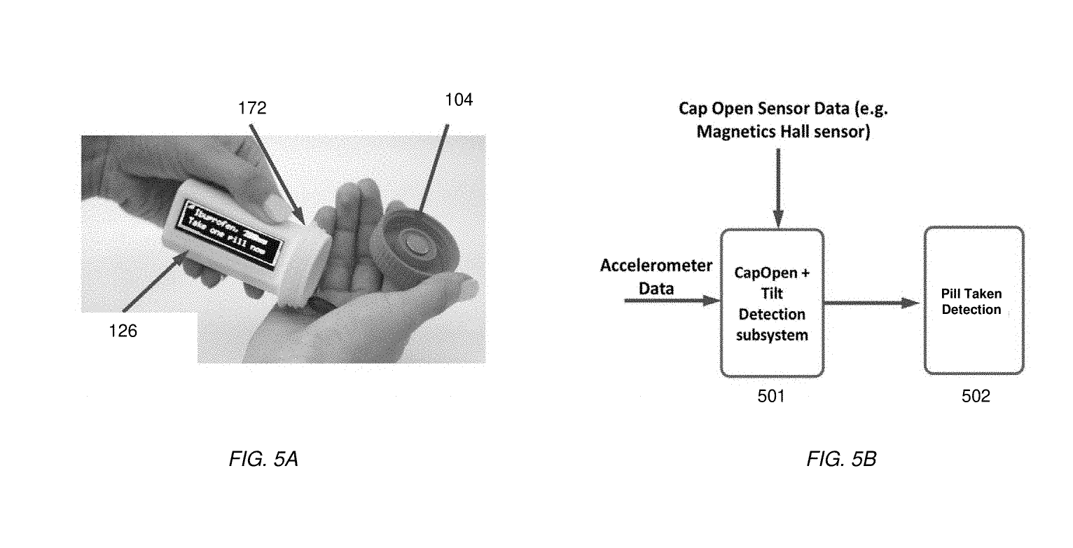

FIGS. 5A and 5B generally illustrate the operation of pill dispensing detection according to one embodiment of the present design. FIG. 5A shows the bottle cap 104, including a magnet 128 separated from the bottle 102. This representation shows the underside of the cap and interior to the cap interior thread 116 is the magnet mounting collar 142 that mounts the magnet 128 onto the underside of the bottle cap 104. With the magnet 128 fixed to the cap, the Hall detector 172 can detect when the cap is removed and placed back onto the bottle 102. The Hall detector 172 is shown in FIG. 1B and is located in the double wall cavity 140. In practice, the display 138 may show an indication, such as “Ibuprofen 200 mg Take one pill now” and the user may pick up the bottle 102 and remove the cap, thus removing the magnet from the bottle. This in turn causes the magnetic sensor or Hall sensor 172 in the bottle to detect cap removal and an accelerometer 126 also located in bottle 102 detects tilt when the user tilts the bottle to retrieve her ibuprofen pill.

FIG. 5B generally illustrates the detection of bottle cap removal, i.e. removal of the bottle cap 104 from the bottle 102. Operation begins at point 501, wherein the system receives accelerometer data and cap open data, such as by sensing from the Hall sensor 172. The processor in the bottle 102 senses cap open and tilt and indicates a single pill has been taken at point 502. In general, tilt more than 90 degrees from vertical, sensed by the accelerometer, indicates pouring pills out of bottle 102. It is assumed that the patient takes the recommended medication; additional attributes described herein may be used to ensure the proper number of pills have been taken and/or the number of pills remaining in the bottle 102 is determined.

While FIGS. 5A and 5B emphasize use of the Hall sensor 172 and accelerometer 126, other sensors may be employed in determining when a pill has been received or removed. For example, an optical emitter and detector 132 may be used with the bottle 102 with an optical reflector 134 provided on the bottle cap 104. Using such a detector and reflector combination, the processor in the bottle 102 may sense removal of the cap. Other sensors as described herein may also or alternately be employed in any combination.

Quantifying/Estimating Pill Amounts

Pills may be counted using different device provided with the present design. It is to be understood that some or all of the sensors disclosed may be provided in any combination. FIG. 1B shows surface acoustic sensor 124, used to detect the amplitude and frequency resonating when shaking the contents of the bottle. The frequency spectrum and amplitude of the acoustic signal is a relatively strong indication of the number of pills remaining in the bottle 102. The more pills when the bottle is shaken results a more significant spectrum represented in the lower frequency range. In other words, a larger amplitude at a lower frequency indicates a larger number of pills. The bottle and more specifically the processor provided may include a spectrum analyzer. A spectrum analyzer analyzes the acoustic frequency spectrum when, for example, the bottle is shaken and can employ processing to determine the number of pills remaining in the bottle.

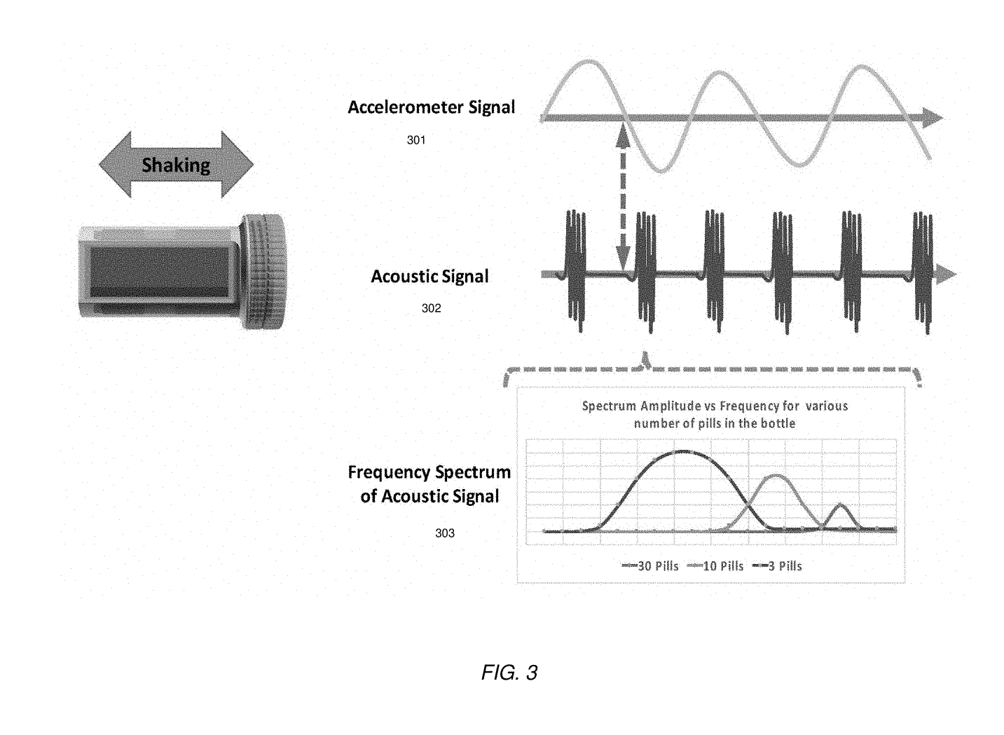

FIG. 3 shows a standard “horizontal” orientation of bottle 102 and shaking of the bottle in a side-to-side motion, such as a “horizontal” shaking motion. FIG. 3 illustrates a resultant accelerometer signal 301 and an acoustic signal 302 and the result of spectral analysis with a frequency spectrum of the resultant acoustic signal at frequency spectrum graph 303. Pills may be counted or determined by employing a surface acoustic sensor together with accelerometer 126 to determine the accelerometer signal 301 produced by the shaking of the bottle system 100. Acoustic signal 302 represents the acoustic signal produced and received when bottle 102 is shaken and the pills strike the bottle wall interior 118. Frequency spectrum graph 303 shows the spectrum amplitude versus the frequency indicating the frequency spectrum of shaking, from which it can be determined the number of pills likely present in the bottle.

Thus the user or another individual can employ a surface acoustic sensor with accelerometer to determine the number of pills inside the bottle by simply shaking the bottle. The processor in the bottle receive acoustic and acceleration signals, where shaking causes the pills to strike against the side of the bottle, resulting in an acoustic frequency and amplitude as well as an acceleration of the shaking. The accelerometer 126 receives the accelerations of the shaking performed. The frequency and amplitude of the totality of the pills present and striking the interior of bottle 102 is received by the surface acoustic sensor. The processor determines a graph similar to that shown as frequency spectrum graph 303 by processing both the acoustic data and the accelerometer data and may estimate the number of pills remaining.

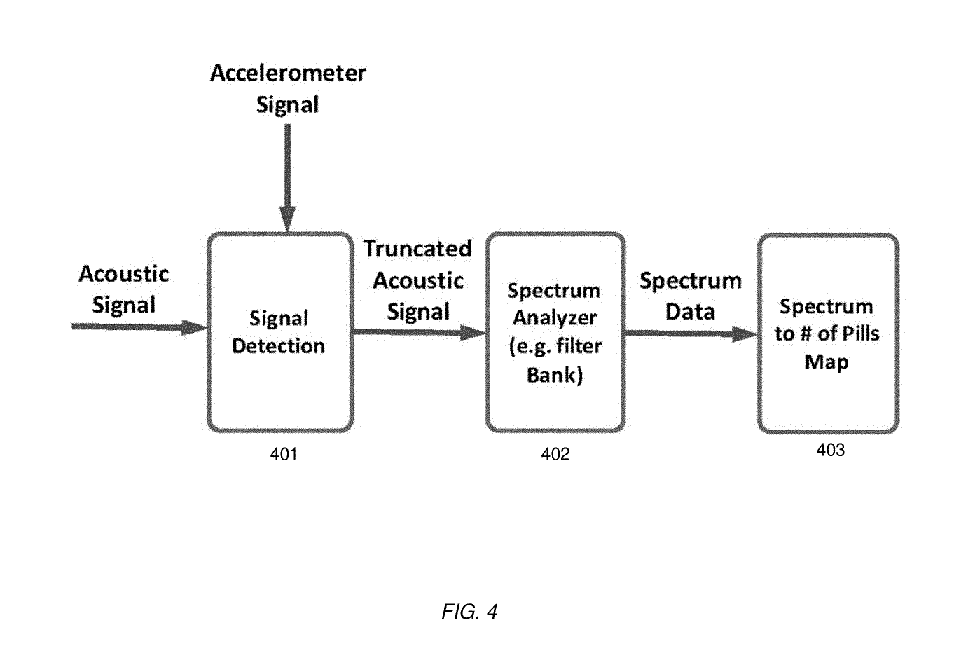

FIG. 4 is a general schematic representation of information processing when the surface acoustic sensor is used with accelerometer 126. From FIG. 4, the user shakes the bottle 102 and an accelerometer signal and acoustic signal is received, shown as signal detection 401. The acoustic signal may be attenuated or otherwise processed or truncated, with the signal detection and processing performed by the processor within the bottle or in one alternate embodiment at a remote location such as at an offline server or computing device. Point 402 represents a spectrum analyzer either provided with the processor in bottle 102 or outside on a remote computing device, where spectrum analyzer 402 may include a filter bank and analyzes both the acoustic signal received and the acceleration of the shaking and provides spectrum data. The combined acoustic and acceleration data determines an estimated number of pills in the bottle 102.

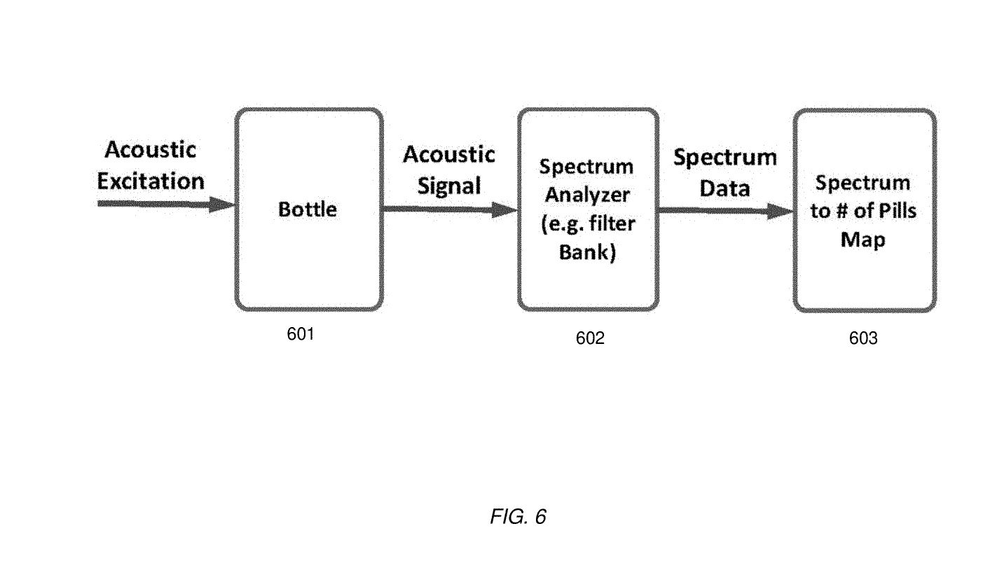

An additional method of pill counting employs acoustic excitation, such as a buzzing sound, with the acoustic generator 146. FIG. 6 illustrates the path by which pills are counted by use of the acoustic generator 146. Such generators can produce an acoustic signal, which is then recorded and subsequently processed to determine the peak frequency corresponding to resonant frequency. The system may determine peak frequency and the resonant frequency tends to be inversely proportional to the number of pills in the bottle. The air volume is approximately proportional to the number of pills in the bottle. Using these proportions allows for a relatively accurate assessment of the number of pills remaining the in the bottle.

FIG. 6 illustrates a bottle 601 that receives acoustic excitation, such as shaking and the surface acoustic sensor and processor operate to provide an acoustic signal. The processor may employ a spectrum analyzer, potentially including a filter bank, shown as spectrum analyzer 602, and produces spectrum data similar to that shown in FIG. 3 as frequency spectrum graph 303. Element 603 represents the processor mapping the spectrum received to a number of pills using known attributes of frequency spectrum, the size and attributes of the bottle, and the size and attributes of the pills and/or other objects within the bottle. Form this, the system may also employ accelerometer data, if available, and may determine an estimate of the number of pills.

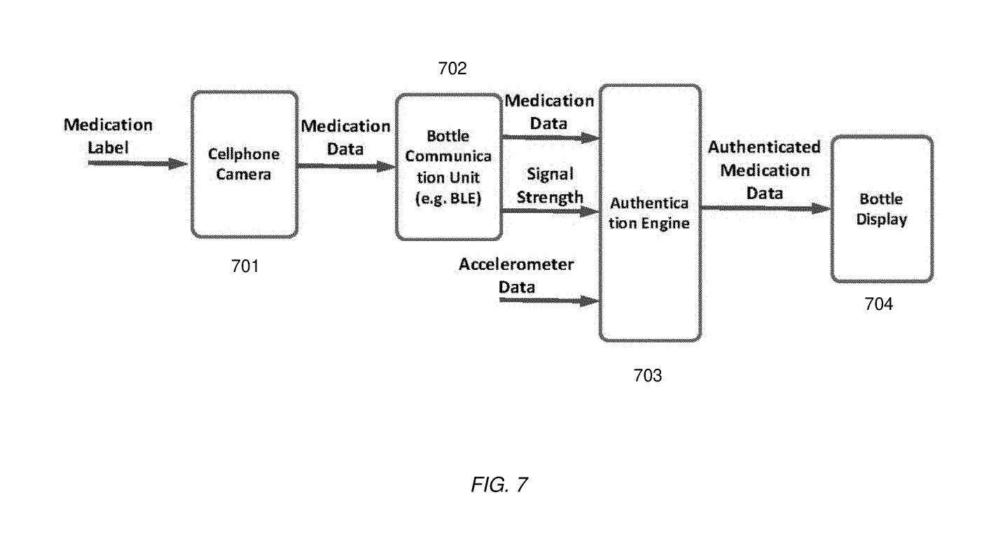

FIG. 7 represents, in general, transferring medication data securely to the bottle in one embodiment. From FIG. 7 a visual recording device 701, such as a cellphone camera, may capture a visual representation of a medication label, e.g. Ibuprofen, 200 mg, with other information. The information and/or visual representation may be provided by the visual recording device 701 to a communication unit provided in or with bottle 102, which may employ the processor to determine information such as medication data and other relevant information, including but not limited to signal strength between the visual recording device or other transmitter and the bottle transmitter, patient name, dosage of the pills, prescriber name, pharmacy or provider, and so forth. Accelerometer data may also be provided to the processor, and the processor may include an authentication engine 703 that authenticates the medication information. For example, if Mrs. Smith is to receive a prescription for 200 mg of Prescription C, because she has previously received such a prescription or is otherwise known to the processor to require prescription C, the presence on the visually captured label that the prescription is for Mrs. Smith and for Prescription C can be verified by the authentication engine 703 of the processor. The authenticated medicated data may then he displayed on the bottle. Accelerometer or other available data may be employed to verify the prescription has been inserted into the bottle, and other appropriate functions and calculations may be performed to display appropriate information.

Pill Dispensing Detection

The present system may also detect when a medication dose has been dispensed. The present bottle system 100 includes multiple methods by which pill dispensing can be detected. The present system may, for example, detect pill dispensing using accelerometer 126. When the bottle is moved from a static position and tilted so that the pills are rolled and shifted to outside of the bottle, the accelerometer 126 detects such movement, and for example when excess of 90 degrees from vertical, such acceleration is strongly indicative of dispensing of a medication or pill. The present system checks if the cap is open using a magnetic sensor or Hall sensor 172 together with a Hall switch on the bottle. If the bottle is tilted more than a certain angle, such as more than 90 degrees from vertical, the system detects the tilt and a “pill dispense” event is marked.

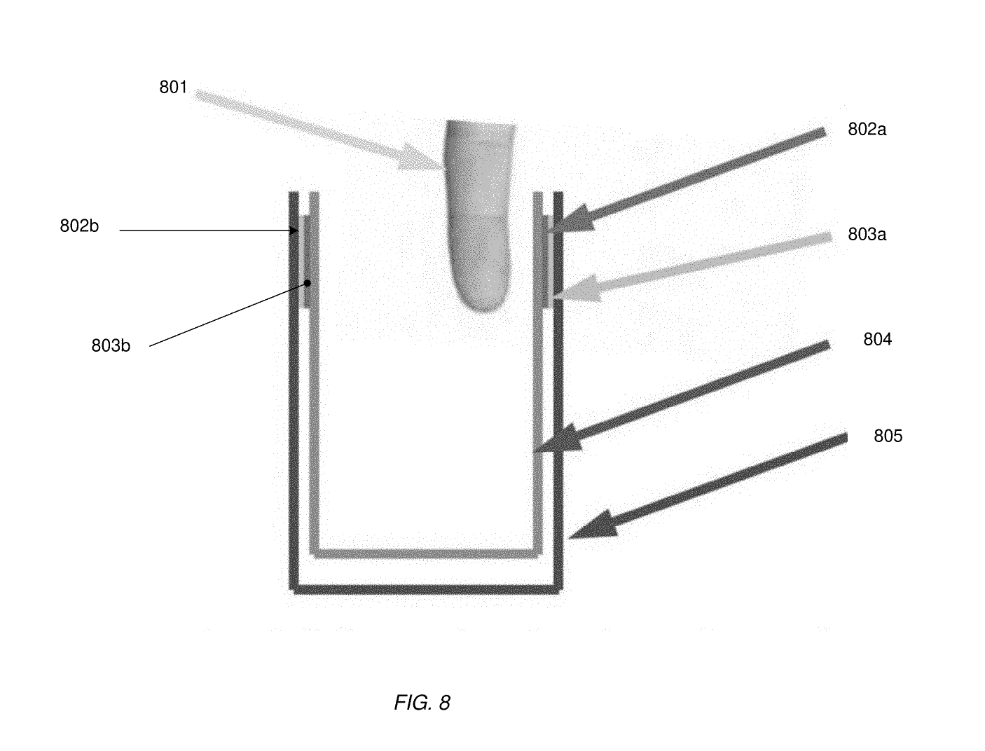

Another embodiment for detecting a pill dispensing event is that of taking pills out of bottle use of a putting a finger inside the bottle and moving the pill out of the bottle. The detection mechanism here detects this scenario by checking the finger existence using capacitive sensing electrodes 120. Shown in FIG. 8 is the double wall cavity 140 with the capacitive sensing electrodes 120 provided inside bottle 102. The capacitive sensing electrodes are shielded by the outer shield 122 to prevent false detection events. Further, a shield layer may be implemented on the outer layer of the touch sensors to make the sensor insensitive to the touching or holding on the outside on the bottle.

FIG. 8 shows an embodiment offering, physical contact authentication. From FIG. 8, finger 801, representing any part of a user or individual entering the bottle portion of bottle 102, is shown together with at least one capacitive sensing electrode shown as 802a and 802b, While shown as two capacitive electrodes, a single ring-shaped or partial ring-shaped electrode may be employed, and FIG. 8 represents a cross sectional view, and thus sensors 802a and 802b may be two parts of a single unitary sensor element. Also provided are outer shield(s) 803a and 803b, which are similarly either a unitary construction or multiple components, representing outer shielding for the capacitive sensing electrodes 802a and 802b. The inner bottle packaging layer 804 and outer layer packaging sensor 805 is also provided. The user reaching into the bottle may be sensed by sensors 802a and 802b and a “pill removal” event triggered, with a resultant decrease in the number of pills considered to be present in the bottle.

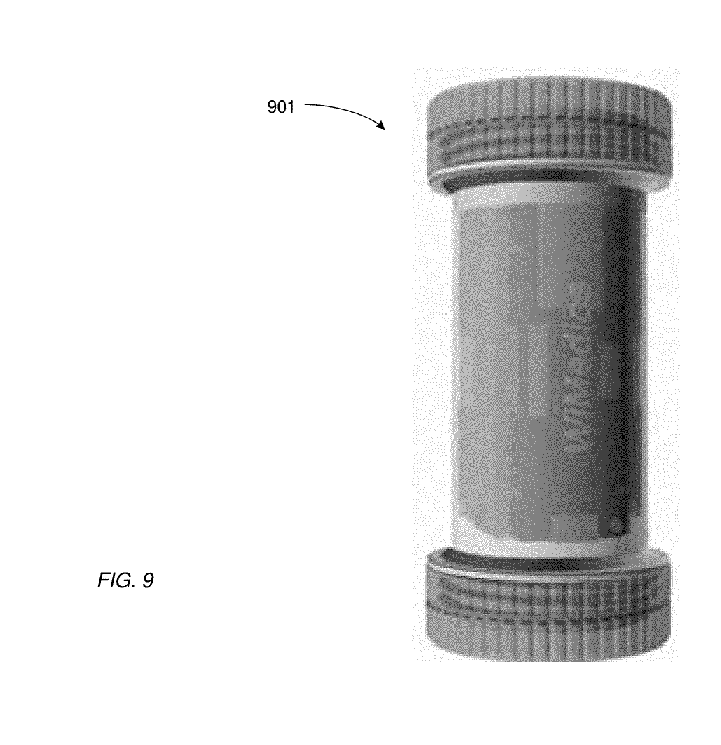

FIG. 9 is a further embodiment representing a dual pill bottle design offering ability to maintain and distribute two different types of pills. From FIG. 9, there is shown double ended bottle 901 that can house two types of pills simultaneously. Double ended bottle 901 includes two lids and pill storage is partitioned into two separate spaces. In this embodiment, the electronics may be shared between the two spaces, but two sets of electronics may alternately be employed. The accelerometer can be used to detect the orientation of the double ended bottle 901, and when a user is distributing one pill from one end versus a second pill from the second end. The display provided with double ended bottle 901 may provide information relevant to the side of the bottle being used, potentially based on accelerometer information. Two cap opening sensors (such as magnetic sensors, may be located on each end to detect which side of the pill container is opened.

The accelerometer can also be used as a part of power wake up scheme. When the user moves the bottle, the accelerometer can detect the move and wake the electronics from a sleep state. The display plus one or more LEDs in the system and a sound generating component (such as piezo electric buzzer) may be used to generate feedback and also display the medication information as well as alerts and other relevant information.

Thus a smart medication bottle system is provided. The smart medication bottle system includes a bottle, a thread positioned on one end of the bottle, and a closed bottle base on the other end. The threading closes two walls of the bottle creating a double walled cavity between an external bottle wall and an internal bottle wall, threading onto the threaded end of the bottle, is a cap which has corresponding thread. Through the threading is the aperture of the bottle, allowing pills to be placed internally of the bottle. The double wall cavity houses the electronics, including all the sensors. The sensors are able to count the pills and can determine when a pill has been dispensed. On one side of the bottle there is placed a user interface display. This display will inform a patient regarding the bottle contents. The bottle is capable of full communication with a smartphone.

Further, according to the present design, there is provided an apparatus comprising a bottle portion comprising electronics including an accelerometer, an acoustic sensor, and a processor, the bottle portion configured to hold a quantity of pills, and a cap portion joinable to the bottle portion. The accelerometer and processor are configured to determine at least one of an acceleration of shaking of the bottle portion and cap portion, and a tilting of the bottle portion to a predetermined orientation evidencing removal of at least one pill from the bottle portion. The acoustic sensor, accelerometer, and processor are configured to determine a frequency spectrum of an acoustic signal obtained when the bottle portion and cap portion are shaken and determine an estimated quantity of pills based on the frequency spectrum.

According to another embodiment of the present design, there is provided a medication container bottle system comprising a bottle portion comprising a display and a processor and configured to receive and maintain a quantity of pills, and a cap portion joinable to the bottle portion, wherein the cap portion and bottle portion comprise at least two of (a) an accelerometer provided with the bottle portion, (b) an acoustic sensor provided with the bottle portion, (c) a magnet provided with the cap portion and a corresponding magnetic sensor provided with the bottle portion, (d) an optical transmitter/receiver and a corresponding optical reflector provided with the bottle portion, and (e) at least one capacitive sensing electrode provided proximate an opening on the bottle portion.

According to a further embodiment, there is provided a bottle system, comprising a bottle portion comprising electronics including a display, an accelerometer, an acoustic sensor, and a processor, the bottle portion configured to hold a quantity of pills, and a cap portion joinable to the bottle portion. The accelerometer and processor are configured to determine an acceleration of shaking of the bottle portion and cap portion with the quantity of pills contained therein, and further wherein the acoustic sensor, accelerometer, and processor are configured to determine a frequency spectrum of an acoustic signal obtained when the bottle portion and cap portion are shaken and determine an estimated quantity of pills based on the frequency spectrum.

What has been described above includes examples of one or more embodiments. It is, of course, not possible to describe every conceivable combination of components or methodologies for purposes of describing the aforementioned embodiments, but one of ordinary skill in the art may recognize that many further combinations and permutations of various embodiments are possible. Accordingly, the described embodiments are intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An apparatus, comprising:
a bottle portion comprising electronics including an accelerometer, an acoustic sensor, and a processor, the bottle portion configured to hold a quantity of pills; and
a cap portion joinable to the bottle portion;
wherein the accelerometer and processor are configured to determine at least one of:
an acceleration of shaking of the bottle portion and cap portion; and
a tilting of the bottle portion to a predetermined orientation evidencing removal of at least one pill from the bottle portion;
and further wherein the acoustic sensor, accelerometer, and processor are configured to determine a frequency spectrum of an acoustic signal obtained when the bottle portion and cap portion are shaken and determine an estimated quantity of pills based on the frequency spectrum.

2. The apparatus of claim 1, wherein the cap portion and the bottle portion comprise a magnet and a magnetic sensor, and further wherein the processor is employed with the magnet and magnet sensor to determine when the cap portion has been removed from the bottle portion.

3. The apparatus of claim 1, wherein the cap portion and the bottle portion comprise an optical transmitter/receiver and an optical reflector, and wherein the processor is employed with the optical transmitter/receiver and optical reflector to determine when the cap portion has been removed from the bottle portion.

4. The apparatus of claim 1, further comprising a transmitter configured to transmit information from the apparatus to a remote device.

5. The apparatus of claim 1, wherein the bottle portion comprises a display configured to operate with the processor to display information relevant to the contents of the apparatus.

6. The apparatus of claim 1, further comprising at least one capacitive sensing electrode positioned within walls of the bottle portion and connected to the processor to determine when a portion of a user has entered the bottle portion.

7. The apparatus of claim 1, wherein the processor employs spectral analysis of acoustic signals received.

8. A medication container bottle system comprising:
a bottle portion comprising a display and a processor and configured to receive and maintain a quantity of pills; and
a cap portion joinable to the bottle portion;
wherein the cap portion and bottle portion comprise at least two of:
(a) an accelerometer provided with the bottle portion;
(b) an acoustic sensor provided with the bottle portion;
(c) a magnet provided with the cap portion and a corresponding magnetic sensor provided with the bottle portion;
(d) an optical transmitter/receiver and a corresponding optical reflector provided with the bottle portion; and
(e) at least one capacitive sensing electrode provided proximate an opening on the bottle portion.

9. The medication container bottle system of claim 8, wherein the cap portion and the bottle portion comprising the acoustic sensor employs the acoustic sensor to determine a frequency spectrum of an acoustic signal obtained when the bottle portion and cap portion are shaken and determine an estimated quantity of pills based on the frequency spectrum.

10. The medication container bottle system of claim 9, wherein the processor employs spectral analysis of acoustic signals received.

11. The medication container bottle system of claim 8, wherein the cap portion and the bottle portion comprising the at least one capacitive sensing electrode employs the at least one capacitive sensing electrode within walls of the bottle portion and connected to the processor to determine when a portion of a user has entered the bottle portion.

12. The medication container bottle system of claim 8, wherein the cap portion and the bottle portion comprising the magnet and magnet sensor employs the magnet and magnet sensor to determine when the cap portion has been removed from the bottle portion.

13. The medication container bottle system of claim 8, wherein the cap portion and the bottle portion comprising the optical transmitter/receiver and optical reflector employs the optical transmitter/receiver and optical reflector to determine when the cap portion has been removed from the bottle portion.

14. The medication container bottle system of claim 8, wherein the cap portion and the bottle portion comprising the accelerometer employs the accelerometer to determine orientation of the bottle portion to determine a pill dispensing event.

15. A bottle system, comprising:
a bottle portion comprising electronics including a display, an accelerometer, an acoustic sensor, and a processor, the bottle portion configured to hold a quantity of pills; and
a cap portion joinable to the bottle portion;
wherein the accelerometer and processor are configured to determine an acceleration of shaking of the bottle portion and cap portion with the quantity of pills contained therein, and further wherein the acoustic sensor, accelerometer, and processor are configured to determine a frequency spectrum of an acoustic signal obtained when the bottle portion and cap portion are shaken and determine an estimated quantity of pills based on the frequency spectrum.

16. The bottle system of claim 15, wherein the accelerometer and processor are further configured to determine a tilting of the bottle portion to a predetermined orientation evidencing removal of at least one pill from the bottle portion.

17. The apparatus of claim 1, wherein the acoustic sensor is a surface acoustic sensor.

18. The medication container bottle system of claim 8, wherein the acoustic sensor is a surface acoustic sensor.

19. The bottle system of claim 15, wherein the acoustic sensor is a surface acoustic sensor.
